SEQUENCE LISTING (21 sequences)

SEQ ID NO: 1 - protein, 448 aa, synthetic construct
MGSSHHHHHHSSGLVPRGSHMVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKEFGTENLYAVLQSGFRGSGGSMVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 2 - protein, 448 aa, synthetic construct
MGSSHHHHHHSSGLVPRGSHMVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKGTAVLQSGFRAVLQSGFRAAAMVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 3 - protein, 464 aa, synthetic construct
MGSSHHHHHHSSGLVPRGSHMVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKGTAVLQSGFRAVLQSGFRAVLQSGFRAVLQSGFRAAAMVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 4 - protein, 496 aa, synthetic construct
MGSSHHHHHHSSGLVPRGSHMVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKGTAVLQSGFRAVLQSGFRAVLQSGFRAVLQSGFRAVLQSGFRAVLQSGFRAVLQSGFRAVLQSGFRAAAMVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 5 - protein, 501 aa, synthetic construct
MGSSHHHHHHSSGLVPRGSHMVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKGTTVLQAVTFQSAVKLQNTVLQAVLQSGFRVRLQAAVLQSGFRTVLQAVTFQSAVKLQNTVLQAVLQSGFRAAAMVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 6 - protein, 448 aa, synthetic construct
MGSSHHHHHHSSGLVPRGSHMVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKGTAVLASGFRAVLQSGFRAAAMVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 7 - protein, 448 aa, synthetic construct
MGSSHHHHHHSSGLVPRGSHMVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKGTAVLQSGFRAVLASGFRAAAMVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 8 - protein, 448 aa, synthetic construct
MGSSHHHHHHSSGLVPRGSHMVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKGTAVLASGFRAVLASGFRAAAMVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 9 - protein, 448 aa, synthetic construct
MGSSHHHHHHSSGLVPRGSHMVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKGTGSGSGSGSAVLQSGFRAAAMVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 10 - protein, 448 aa, synthetic construct
MGSSHHHHHHSSGLVPRGSHMVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKGTAVLQSGFRGSGSGSGSAAAMVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 11 - protein, 610 aa, synthetic construct
MGSSHHHHHHHHHHSSGLVPRGSHGSGSGSQVQLQESGGGSVQAGGSLRLSCAVSGYSYCNYDLGWFRQAPGKEREGIATIDSDGNTYYVDSVKGRFTISQDNAKNTVALQMNSLKPEDTAMYYCKVGSIASSVPEVSCPPSAPFGYWGQGTQVTVSSGSGSGSGSGSGSGSGSGSGSGSGSMVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKGTAVLQSGFRAVLQSGFRAAAMVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 12 - protein, 598 aa, synthetic construct
MGSSHHHHHHHHHHSSGLVPRGSHGSGSGSQVQLQESGGGSVQAGGSLRLSCAVSGFIATSCALGWFRQAPGKEREGIATITTDGTTYYVDSVKGRFTISQDNAKNTVALQMNSLKPEDTAMYYCKLCCSGQYCAWGQGTQVTVSSGSGSGSGSGSGSGSGSGSGSGSGSMVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKGTAVLQSGFRAVLQSGFRAAAMVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 13 - protein, 600 aa, synthetic construct
MGSSHHHHHHSSGLVPRGSHMVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKGTAVLQSGFRAVLQSGFRAAAMVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAGSGSGSGSGSGSGSGSGSGSGSGSQVQLQESGGGSVQAGGSLRLSCAVSGYSYCNYDLGWFRQAPGKEREGIATIDSDGNTYYVDSVKGRFTISQDNAKNTVALQMNSLKPEDTAMYYCKVGSIASSVPEVSCPPSAPFGYWGQGTQVTVSS

SEQ ID NO: 14 - protein, 588 aa, synthetic construct
MGSSHHHHHHSSGLVPRGSHMVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKGTAVLQSGFRAVLQSGFRAAAMVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAGSGSGSGSGSGSGSGSGSGSGSGSQVQLQESGGGSVQAGGSLRLSCAVSGFIATSCALGWFRQAPGKEREGIATITTDGTTYYVDSVKGRFTISQDNAKNTVALQMNSLKPEDTAMYYCKLCCSGQYCAWGQGTQVTVSS

SEQ ID NO: 15 - protein, 8 aa, synthetic construct
AVLQSGFR

SEQ ID NO: 16 - protein, 8 aa, synthetic construct
AVLASGFR

SEQ ID NO: 17 - protein, 8 aa, synthetic construct
GSGSGSGS

SEQ ID NO: 18 - protein, 16 aa, synthetic construct
AVLQSGFRAVLQSGFR

SEQ ID NO: 19 - protein, 32 aa, synthetic construct
AVLQSGFRAVLQSGFRAVLQSGFRAVLQSGFR

SEQ ID NO: 20 - protein, 64 aa, synthetic construct
AVLQSGFRAVLQSGFRAVLQSGFRAVLQSGFRAVLQSGFRAVLQSGFRAVLQSGFRAVLQSGFR

SEQ ID NO: 21 - protein, 62 aa, synthetic construct
TVQAVTFQSAVLQNTVQAVQSGFRVRQAAVQSGFRTVQAVTFQSAVLQNTVQAVQAVQSGFR

(19) United States
(12) Patent Application Publication
(10) Pub. No.: US 2025/0019399 A1
(43) Pub. Date: Jan. 16, 2025

(54) BRET-BASED MPRO BIOSENSOR WITH AN INCREASED RATE OF CLEAVAGE

(71) Applicant: Qatar Foundation for Education, Science and Community Development, Doha (QA)

(72) Inventors: Anupriya M. Geethakumari, Doha (QA); Kabir H. Biswas, Doha (QA)

(21) Appl. No.: 18/750,049

(22) Filed: Jun. 21, 2024

Related U.S. Application Data
(60) Provisional application No. 63/522,298, filed on Jun. 21, 2023.

(51) Int. Cl. C07K 7/06; C07K 16/10

(52) U.S. Cl. CPC C07K 7/06; C07K 16/1003; C07K 2317/569; C07K 2319/30; C07K 2319/50; C07K 2319/60

(57) ABSTRACT

Example systems, methods, and apparatus are disclosed herein for a BRET-based Miro biosensor including an mNeonGreen (mNG) reporter protein, a NanoLuc (NLuc) reporter protein, and x repeats of an N-terminal autocleavage peptide sequence of M^{pro}. The x repeats of an N-terminal autocleavage peptide sequence of M^{pro} are located between the mNG reporter protein and the NLuc reporter protein.

### PRIORITY CLAIM AND CROSS-REFERENCE TO RELATED APPLICATIONS

The present application claims priority to U.S. Provisional Patent Application No. 63/522,298, filed on Jun. 21, 2023, and entitled BRET-BASED MPRO BIOSENSOR WITH AN INCREASED RATE OF CLEAVAGE, the contents of which are hereby incorporated by reference in their entirety.

### SEQUENCE LISTING

This application contains a sequence listing having the filename 1959919-00008 Sequence Listing.xml, which is 26 KB in size, and was created on Sep. 16, 2024. The entire content of this sequence listing is incorporated herein by reference.

### BACKGROUND

The emergence of SARS-COV-2 and its variants has posed a massive healthcare problem. In this regard, pharmacological targeting of the main protease (M^{pro}; 3CL^{pro}) appears to be a key therapeutic strategy to reduce the COVID-19 symptoms and therefore, there is a significant need for the development of such inhibitors. Therefore, a number of assays have been developed to aid in the discovery of such inhibitors. Recently, a genetically encoded, Bioluminescence Resonance Energy Transfer (BRET)-based M^{pro} biosensor has been reported.

Severe Acute Respiratory Syndrome Coronavirus-2 (SARS-COV-2), a member of the beta-coronavirus family and the causative agent of COVID-19 (Coronavirus Disease of 2019), has emerged as a major health and economic threat in the current times. The infection cycle of the virus is initiated by the binding of the virus to the host receptor, angiotensin-converting enzyme 2 (ACE2) expressed on the epithelial cell membranes of the human host in a multitude of organs including the lungs, nose, and the gut. The viral binding is mediated by the S1 spike protein present on the viral surface. The binding of the virus is followed by endocytosis-mediated internalization and proteolytic cleavage of the S1 spike protein by the host protease TMPRSS2, making the virus competent for fusion with the host membrane. Following viral-host cell membrane fusion, the viral genome is released in the host cytosol where the positive-sense RNA-genome gets translated into viral proteins. These include both structural as well as non-structural proteins that are essential for viral replication and further infection. Of particular interests are the Open Reading Frame 1a (ORF1a) and ORF 1b that together represent two-thirds of the SARS-COV-2 genome. These ORFs are translated into two polyproteins referred to as polyprotein 1a (pp1a) and pp1ab, respectively. These polyproteins consist of the non-structural proteins (nsp) 1 to 11 and 1 to 16, respectively. These polyproteins are proteolytically processed to generate functionally active proteins that are necessary for viral replication by the viral proteases, main protease M^{pro} (also referred to nsp5 or 3-chymotrypsin-like cysteine protease (3CLpro) or main proteinase) and papain-like protease (PL^{pro}). Importantly, M^{pro} is a cysteine protease that cleaves the pp1ab at 11 conserved sites (recognition site—LQ↓SAG), including its own autocleavage at the N-and C-termini. It is active in a homodimeric form, and each protomer has three domains-domain I, II and III. The substrate-binding site with Cys145-His41 catalytic dyad is present in a wide cleft formed between the domains I and II whereas domain III is responsible for protein dimerization.

The tremendous threat posed by COVID-19 has resulted in numerous efforts to develop novel therapeutic strategies to control and eradicate this pandemic. These include both the development of novel pharmacological anti-SARS-COV-2 agents and repurposing clinically approved drugs that may either directly or indirectly reduce viral infections. Additionally, significant efforts have been directed towards effective vaccine development using through the traditional methods such as using either whole attenuated virus or viral structural proteins as well as novel RNA-based strategies. However, the frequent emergence of SARS-COV-2 variants/lineages with mutations in the SI spike or other proteins providing increased infection and immune evasion potential to the virus has raised concerns regarding their efficacy. Therefore, alternatives to vaccines are suggested to be required for the successful elimination of the pandemic. In this regard, M^{pro} is considered an appealing target for designing anti-SARS-COV-2 agents since none of the human proteases have similar cleavage sequence specificity. Indeed, a number of structural, biophysical, and biochemical studies have reported the specific pharmacological targeting of M^{pro} using both de novo drug discovery as well as repurposing of already available drugs, although with limited success. In this regard, the development of facile technologies will accelerate further drug discovery programs against M^{pro}, and thus, SARS-COV-2.

M^{pro} has been typically studied in vitro using fluorescently labeled cognate peptides that show reduced fluorescence resonance energy transfer (FRET) upon cleavage of the peptide is cleaved by the enzyme. Such assays have been utilized for the identification of Boceprevir, GC376, and calpain inhibitors II, XII as potent inhibitors of SARS-COV-2 Mpro13. M^{pro} activity has been determined through direct visualization of cleaved proteins in sodium dodecyl sulfate-polyacrylamide gel electrophoresis. While convenient, these assays cannot be implemented in living cells. Additionally, double labeling of the peptide during synthesis is significantly costly. These have led to the development of genetically encoded biosensors that can be used in living cells with a significantly reduced assay cost and possible gain in sensitivity. Genetically encoded biosensors, either based on fluorescence or bioluminescence, have seen significant usage in assessing a variety of cellular processes including cellular signaling events, protein conformational changes, and protein-protein interactions including oligomerization in living cells. Specifically, biosensors have been utilized for detecting a number of viruses including HIV, Hepatitis, Zika, Dengue, Ebola, and Influenza viruses as well as SARS-COV and MERS-COV19. Further, split protein-based biosensors, using either luciferase or GFP as reporters, have also been developed to monitor M^{pro} activity in live cells. In these biosensors, M^{pro}-mediated cleavage of the split protein results in the activation of the reporter protein (luciferase or GFP), which is then measured in microplate readers or quantified from microscopic images. Additionally, M^{pro} activity has also been monitored through gene expression changes as well as through nuclear localization of reporter protein upon proteolytic cleavage.

A need therefore exists for a BRET-based M^{pro} biosensor with an increased rate of cleavage.

### SUMMARY

Example systems, methods, and apparatus are disclosed herein for a BRET-based Mpro biosensor with an increased rate of cleavage.

The disclosed invention includes synthetic M^{pro} cleavage sequences to be utilized for generating BRET-based M^{pro} biosensors displaying increased M^{pro}-mediated proteolytic cleavage rate and show that a biosensor containing 2×cleavage site performs better as a M^{pro} substrate. These sequences included 2×, 4× and 8× repeats of the M^{pro} N-terminal (Nter) cleavage sequences and a combination of M^{pro} cleavage sequences containing a total of 12 cleavage sites. Secondary structure predictions as well three-dimensional structural modeling indicated predominantly alpha-helical structure for both the synthetically designed sequences as well as the original M^{pro} Nter cleavage (1×) sequence. Gaussian accelerated molecular dynamics (GaMD) simulations of the synthetic M^{pro} cleavage sequences revealed a dynamic nature of the cleavage sequences, which is critical for their efficient cleavage, and a relatively short end-to-end distances, which is required for high BRET. Live cell and in vitro assays revealed a cleavage sequence length-dependent resonance energy transfer, except for the 12×-syn cleavage site and an enhanced rate of cleavage for the M^{pro} biosensor containing 2× cleavage sequences. The inclusion of a M^{pro}-binding, but non-inhibiting, NB2E3 nanobody at the N-terminal further increased the cleavage rate of the 2× cleavage sequence containing the M^{pro} biosensor. The 2× M^{pro} biosensor will find utility in both the development M^{pro} proteolytic activity assays that can be employed for drug discovery applications as well as functional genomics application in characterization impact of mutations in the M^{pro} protein.

In light of the disclosure herein, and without limiting the scope of the invention in any way, in a first aspect of the present disclosure, which may be combined with any other aspect listed herein unless specified otherwise, a BRET-based M^{pro} biosensor including an mNeonGreen (mNG) reporter protein, a NanoLuc (NLuc) reporter protein, and x repeats of an N-terminal autocleavage peptide sequence of M^{pro}. The x repeats of an N-terminal autocleavage peptide sequence of M^{pro} are located between the mNG reporter protein and the NLuc reporter protein.

In a second aspect which may be combined with any other aspect listed herein unless specified otherwise, the x repeats of an N-terminal autocleavage peptide sequence of M^{pro} are two.

In a third aspect which may be combined with any other aspect listed herein unless specified otherwise, the x repeats of an N-terminal autocleavage peptide sequence of M^{pro} are four.

In a fourth aspect which may be combined with any other aspect listed herein unless specified otherwise, the x repeats of an N-terminal autocleavage peptide sequence of M^{pro} are eight.

In a fifth aspect which may be combined with any other aspect listed herein unless specified otherwise, the N-terminal autocleavage peptide sequence of M^{pro} includes a peptide sequence AVLQSGFR (SEQ ID NO:15).

In a sixth aspect which may be combined with any other aspect listed herein unless specified otherwise, the x repeats of an N-terminal autocleavage peptide sequence of M^{pro} are twelve.

In a seventh aspect which may be combined with any other aspect listed herein unless specified otherwise, amino acid residues C-terminal to a critical Gln residue are varied.

In an eight aspect which may be combined with any other aspect listed herein unless specified otherwise, the twelve repeats of an N-terminal autocleavage peptide sequence of M^{pro} comprises SEQ ID NO: 12.

In a nine aspect which may be combined with any other aspect listed herein unless specified otherwise, a BRET-based M^{pro} biosensor including an mNeonGreen (mNG) reporter protein, a NanoLuc (NLuc) reporter protein, x repeats of an N-terminal autocleavage peptide sequence of M^{pro}, a first nanobody, and a second nanobody. The x repeats of an N-terminal autocleavage peptide sequence of M^{pro} are located between the mNG reporter protein and the NLuc reporter protein.

In a tenth aspect which may be combined with any other aspect listed herein unless specified otherwise, the first nanobody is NB1D10.

In an eleventh aspect which may be combined with any other aspect listed herein unless specified otherwise, the first nanobody is a C-terminal fusion.

In a twelfth aspect which may be combined with any other aspect listed herein unless specified otherwise, the first nanobody is an N-terminal fusion.

In a thirteenth aspect which may be combined with any other aspect listed herein unless specified otherwise, the second nanobody is NB2E3.

In a fourteenth aspect which may be combined with any other aspect listed herein unless specified otherwise, the second nanobody is a C-terminal fusion.

In a fifteenth aspect which may be combined with any other aspect listed herein unless specified otherwise, the second nanobody is an N-terminal fusion.

In a sixteenth aspect which may be combined with any other aspect listed herein unless specified otherwise, the x repeats of an N-terminal autocleavage peptide sequence of M^{pro} are two.

In a seventeenth aspect which may be combined with any other aspect listed herein unless specified otherwise, the N-terminal autocleavage peptide sequence of M^{pro} comprises a peptide sequence AVLQSGFR.

In an eighteenth aspect which may be combined with any other aspect listed herein unless specified otherwise, a method of creating a BRET-based M^{pro} biosensor including placing x repeats of an N-terminal autocleavage peptide sequence of M^{pro} between an mNeonGreen (mNG) reporter protein and a NanoLuc (NLuc) reporter protein.

In a nineteenth aspect which may be combined with any other aspect listed herein unless specified otherwise, further including inserting a first nanobody between the nNG reporter protein and the x repeats of an N-terminal autocleavage peptide sequence of M^{pro}, and inserting a second nanobody between the x repeats of an N-terminal autocleavage peptide sequence of M^{pro} and the Nluc reporter protein.

In a twentieth aspect which may be combined with any other aspect listed herein unless specified otherwise, the x repeats of an N-terminal autocleavage peptide sequence of M^{pro} are two.

In a twenty-first aspect of the present disclosure, any of the structure, functionality, and alternatives disclosed in connection with any one or more of FIGS. 1 to 10 may be combined with any other structure, functionality, and alternatives disclosed in connection with any other one or more of FIGS. 1 to 10.

In light of the present disclosure and the above aspects, it is therefore an advantage of the present disclosure to provide users with a BRET-based M^{pro} biosensor with an increased rate of cleavage.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE FIGURES

FIG. 1 is a schematic showing the working principle of a BRET-based M^{pro} biosensor, according to an example embodiment of the present disclosure.

FIG. 2 shows (A) a schematic showing the general design of a BRET-based M^{pro} biosensor; (B) amino acid residue sequences and cartoon representation of predicted three-dimensional structures of various M^{pro} cleavage sequences highlighting all Gln (Q) residues present in the cleavage sequences; and (C-H) Graphs showing backbone (Cα) root-mean-square deviation (RMSD) values (C), backbone (Cα) root-mean-square fluctuation (RMSF) values (D), radius of gyration (RoG) values (E), solvent accessible surface area (SASA) values (F), frequency of the indicated secondary structures (G) and end-to-end distances (H) of the indicated M^{pro} cleavage sequences obtained from 1 μs of GaMD simulations; according to an example embodiment of the present disclosure.

FIG. 3 shows (A-E) Bioluminescence spectra of the 1× (A), 2× (B), 4× (C), 8× (D) and 12×-syn (E) M^{pro} biosensor constructs in the absence of M^{pro} (left panels) and in the presence of M^{pro} (right panels), data shown is from a representative of three independent experiments; and (F,G) graph showing BRET (F) and fluorescence (G) values obtained from cells expressing the indicated M^{pro} biosensor constructs in the absence and in the presence of M^{pro} data shown are mean±s.d. from three independent experiments, with each experiment performed in triplicates, numbers on top in (F) are percentage change in BRET (mean±s.d.) from three independent experiments, with each experiment performed in triplicates, p values shown are adjusted p-values obtained from a two-way ANOVA (adjusted p-values; Sidak's multiple comparisons test; in the absence and presence of M^{pro}); according to an example embodiment of the present disclosure.

FIG. 4 shows (A,B) graphs showing the total bioluminescence of cells expressing the indicated biosensors in the absence (A) and in the presence (B) of M^{pro}; (C,D) Graphs showing BRET of cells expressing the indicated biosensors in the absence (C) and in the presence (D) of M^{pro}; (E) graphs showing % change in BRET measured in cells expressing the indicated M^{pro} biosensors in the presence of M^{pro}; and (F,G) graphs showing the half-time (t_{1/2}) (C) and rate (D) of cleavage of the indicated biosensor in the presence of M^{pro} (obtained from fitting of data show in E). Data shown are mean±s.d. from three independent experiments, with each experiment performed in triplicates, ND, not determined; according to an example embodiment of the present disclosure.

FIG. 5 shows (A,B) graphs showing the total bioluminescence (A) and BRET (B) in cells expressing the 1× (left panels) and 2× (right panels) M^{pro} biosensors in the presence of M^{pro} and GC376; (C,D) Graphs showing the total bioluminescence (C) and BRET (D) in cells expressing the 1× (left panels) and 2× (right panels) M^{pro} biosensors in the presence of M^{pro} and nirmatrelvir. BRET ratios in the absence of M^{pro} are shown in red circles, insets in B and D show % M^{pro} activity in the presence of the indicated concentrations of the inhibitors; and (E,F) Graphs showing log (IC₅₀) of GC376 (E) and nirmatrelvir (F) inhibition of M^{pro} obtained using either the 1× or 2× M^{pro} biosensor, data shown are mean±s.d. from three independent experiments, each performed in triplicates; according to an example embodiment of the present disclosure.

FIG. 6 shows (A,B) graphs showing total bioluminescence over time in cells expressing the indicated 1× or 2× M^{pro} biosensor, either in the absence (A) or in the presence of MPT0; (B,D) graphs showing BRET over time measured in cells expressing the indicated 1× or 2× M^{pro} biosensors, either in the absence (C) or in the presence of M^{pro} (D); (E) graphs showing the percentage change in BRET over time in the presence of M^{pro};and (F,G) graphs showing the half-time (t_{1/2}) (F) and rate (G) of cleavage of the indicated biosensor in the presence of M^{pro} (obtained from fitting of data shown in E), data shown are mean±s.d. from three independent experiments, each performed in triplicates, ND, not determined; according to an example embodiment of the present disclosure.

FIG. 7 shows (A,B) graphs showing the total bioluminescence over time in cells expressing the indicated biosensor, either in the absence (A) or in the presence (B) of M^{pro}; (C,D) graphs showing the BRET over time measured in cells expressing the indicated biosensor, either in the absence (C) or in the presence (D) of M^{pro}; (E) graphs showing the percentage change in BRET over time of the indicated biosensor in the presence of M^{pro}; and (F,G) graphs showing the t_{1/2}(F) and rate of cleavage (G) of the indicated biosensor in the presence of M^{pro} (obtained from fitting of data shown in E), data shown are mean±s.d. from three independent experiments, each performed in triplicates, ND, not determined; according to an example embodiment of the present disclosure.

FIG. 8 shows fluorescence images of HEK 293T cells showing expression of M^{pro} biosensors, according to an example embodiment of the present disclosure.

FIG. 9 shows fluorescence images of HEK293T cells showing expression of mutant 2× M^{pro} biosensors, according to an example embodiment of the present disclosure.

FIG. 10 shows fluorescence images of HEK293T cells showing expression of 2× M^{pro} biosensor constructs containing nanobodies, according to an example embodiment of the present disclosure.

### DETAILED DESCRIPTION

Methods, systems, and apparatus are disclosed herein for a BRET-based M^{pro} biosensor with an increased rate of cleavage.

While the example methods, apparatus, and systems are disclosed herein as BRET-based M^{pro} biosensor with an increased rate of cleavage, it should be appreciated that the methods, apparatus, and systems may be operable with other explorers.

Building on recently reported Bioluminescence Resonance Energy Transfer (BRET)-based M^{pro} biosensor, the disclosed invention generates and characterizes a number of additional biosensor constructs in order to achieve a higher a rate of cleavage. Specifically, the disclosed invention generated biosensors containing 2×, 4× and 8× copies of the M^{pro} N-terminal autocleavage sequence, which has been shown to possess the highest catalytic efficiency for the N-terminal autocleavage sequence, in between mNeonGreen (mNG) and NanoLuc (NLuc) reporter proteins. The disclosed invention follows the principle that a greater number of cleavage sites in the biosensor will lead to an increased effective concentration and therefore, rate of cleavage.

Additionally, the disclosed invention generated a biosensor containing 12× M^{pro} cleavage sites combining most of the 11 M^{pro} cleavage sites present in ppla and pp lab polyproteins in an overlapping fashion to reduce the total length and thus, distance between the BRET reporter proteins. This increases the number of cleavage sites and also allows sampling other cognate M^{pro} cleavage sequences.

Live cell and in vitro assays monitoring M^{pro}-mediated cleavage of the biosensors in the disclosed invention revealed highest rate of cleavage for the biosensor containing 2× M^{pro} N-terminal cleavage sites. Experiments with M^{pro} pharmacological inhibitors, GC376 and nirmatrelvir, revealed a decreased efficacy (higher IC₅₀ values) of the inhibitors in M^{pro}-mediated cleavage of the 2× M^{pro} biosensor in living cells. Additionally, mutation of either the first or the second or both cleavage sites (Q to A) or replacement of the first or the second cleavage site with GS linker sequence revealed a requirement for both cleavage sites to be intact for the increased cleavage rate of the 2× M^{pro} biosensor. In an embodiment, including nanobodies that bind to M^{pro} but do not affect its activity in the 2× M^{pro} biosensor increases cleavage rate of the 2× M^{pro} biosensor when there is N-terminal fusion of the NB2E3 nanobody.

The BRET-based M^{pro} biosensor of the disclosed invention has direct utility in studies related to pharmacological targeting the SARS-COV-2 M^{pro} in living cells mimicking the in vivo conditions, in additional to functional genomics applications such as the determination of the effect of sequence variations in the M^{pro} that are observed in different viral isolates. Further, the biosensors with increased sensitivity can be deployed for point-of-care-testing (POCT) of live SARS-COV-2 infection in patient samples.

### Preparation and Materials

Structural modeling and MD simulation of M^{pro} cleavage sequences: Structural models of the 1×, 2×, 4× and 8× M^{pro} N-terminal and the 12×-syn cleavage sequences were generated using the trRosetta online server (https://yanglab.nankai.edu.cn/trRosetta/) 6, 29, 30 Quality of the structural models were checked using the available tools in the trRosetta server. Molecular dynamics simulations of the M^{pro} cleavage sequences were performed using the Nanoscale Molecular Dynamics (NAMD) software (version 2.13) with the CHARMM36(m) force field. A 2 fs time-step of integration was set for all simulations performed. First, energy minimization was performed on each system for 1000 steps (2 ps). Following energy minimization, the system was slowly heated from 60 K to 310 K at 1 K interval to reach the 310 K equilibrium temperature using a temperature ramp that runs 500 steps after each temperature increment. Following thermalization, temperature was maintained at 310 K using Langevin temperature control and at 1.0 atm using Nose-Hoover Langevin piston pressure control. The system was then equilibrated with 500000 steps (1 ns) using Periodic Boundary Conditions.

The NAMD output structure was then used as an input for Gaussian accelerated molecular dynamics (GaMD) simulation utilizing the integrated GaMD module in NAMD and its default parameters which included 2 ns of conventional molecular dynamics (cMD) equilibration run in GaMD, to collect potential statistics required for calculating the GaMD acceleration parameters, and another 50 ns equilibration run in GaMD after adding the boost potential, and finally GaMD production runs for 1000 ns. Both equilibration steps in GaMD were preceded by 0.4 ns preparatory runs. All GaMD simulations were run at the “dual-boost” level by setting the reference energy to the lower bound, i.e., E=Vmax. One boost potential is applied to the dihedral energetic term and the other to the total potential energetic term. The details for calculating the boost potentials including the equations used have been described previously. The upper limits of standard deviation (SD) of the dihedral and total potential boosts in GaMD were set to 6.0 kcal/mol. All GaMD simulations were performed using the same constant temperature and pressure parameters. For all simulations, short-range non-bonded interactions were defined at 12 Å cut-off with 10 A switching distance, while Particle-mesh Ewald (PME) scheme was used to handle long-range electrostatic interactions at 1 Å PME grid spacing. Trajectory frames were saved every 10,000 steps (20 ps) and trajectory analysis was performed using the available tools in Visual Molecular Dynamics (VMD) software 39. These included Cα root mean squared displacement (RMSD), Ca root mean squared fluctuation (RMSF) for each residue, radius of gyration (RoG), solvent accessible surface area (SASA), secondary structure and end-to-end distance measurements.

M^{pro} biosensor plasmid construct generation: The BRET-based M^{pro} biosensor plasmid constructs were generated by synthesizing nucleotide sequences coding for the 1×, 2×, 4× and 8× repeats N-terminal autocleavage peptides (AVLQSGFR, SEQ ID NO:15) and 12×-syn amino acid residues and subcloning in between the mNG and NLuc genes present in the original M^{pro} biosensor, pmNG-M^{pro}-N-ter-auto-NLuc, plasmid. The 2× M^{pro} biosensor mutants were generated by mutating the critical Gln to Ala (from AVLQSGFR to AVLASGFR (SEQ ID NO: 16)) either in the first (Q262A) or second (Q270A) or both cleavage sequences (double mutant; DM). The 1GS and 2GS mutants of the 2× M^{pro} biosensor were generated by replacing the nucleotide sequence coding for AVLQSGFR with that of GSGSGSGS (SEQ ID NO:17) for the first (1GS) or the second (2GS) M^{pro} cleavage site.

The N- and C-terminal fusion of the NB1D10 and NB2E3 nanobodies in the 2× M^{pro} biosensor were generated by synthesizing gene fragments expressing amino acid residues of the respective nanobody as reported previously, codon optimized for mammalian cell expression, and subcloned into the pmNG-M^{pro}-Nter-auto-2×-NLuc plasmid. All clones were procured from GenScript (Singapore).

Cell culture and transfection: HEK293T cells were grown in Dulbecco's Modified Eagle Medium (DMEM) supplemented with 10% fetal bovine serum, and 1% penicillin-streptomycin and grown at 37° C. in 5% CO2. Transfections were performed with polyethyleneimine (PEI) lipid according to manufacturers' protocol. Briefly, HEK 293T cells seeded onto 96-well white plates were transfected by combining the plasmid DNA (sensor and Mpro), Opti-MEM (Invitrogen; 31985088) and 1.25 μg/well of PEI lipid (Sigma-Aldrich; 408727-100mL) and incubating them at room temperature for 30 min.

Live cell M^{pro} biosensor proteolytic cleavage activity assays: Live cell M^{pro} proteolytic cleavage activity assays were performed by co-transfecting HEK 293T cells with the respective M^{pro} BRET sensor plasmid constructs along with pLVX-EF1alpha-SARS-COV-2-nsp5-2×Strep-IRES-Puro (M^{pro} WT) (Addgene plasmid #141370) in 96-well white flat bottom plates. Post 48 h (or otherwise indicated) of transfection, BRET measurements were performed at 37° C. by the addition of furimazine (Promega, Wisconsin, USA) at a dilution of 1:200. Experiments were performed in triplicate and repeated thrice.

Pharmacological inhibition of M^{pro} in live cells: HEK293T cells were seeded onto the 96-well white plate one day before transfection. The cells were co-transfected with either pmNG-M^{pro}-Nter-auto-NLuc (1×) or pmNG-M^{pro}-Nter-auto-2×-NLuc (2× WT) and pLVX-EF1alpha-SARS-COV-2-nsp5-2×Strep-IRES-Puro (M^{pro} WT) (Addgene plasmid #141370) at a ratio of 1:5. The transfected cells were concurrently treated with GC376 (GC376 Sodium; AOBIOUS-AOB36447; stock solution prepared in 50% DMSO at a concentration of 10 mM) or nirmatrelvir (Sigma; 4 mM stock solution in DMSO). BRET values were measured by adding furimazine (Promega, Wisconsin, USA) at a 1:200 dilution at 16 h post-transfection. The percentage activity was calculated after normalizing the BRET ratio with the negative control (No M^{pro}). The experiments were conducted in triplicates for each construct and repeated thrice.

M^{pro} biosensor lysate preparation and in vitro assay: HEK 293T cells expressing the respective BRET sensor were washed with chilled Dulbecco's Phosphate-Buffered Saline (DBPS) 48 h post transfection and lysed in a buffer containing 50 mM HEPES (pH 7.5), 50 mM NaCl, 0.1% Triton X-100, 1 mM Dithiothreitol (DTT) & 1 mM ethylenediamine tetra acetic acid (EDTA) on ice. Cell lysates were collected in a 1.5 mL eppendorf tubes and centrifuged at 4° C. for 1 h at 14,000 rotations per min (RPM) following which supernatant was collected and stored at −80° C. until further usage. The lysate containing Nluc BRET sensor were mixed with recombinantly purified SARS-COV M^{pro} (3CL Protease, Recombinant from E. coli; NR-700; BEI Resources, NIAID, NIH; stock solution of the protein was prepared by dissolving the lyophilized protein in 50 μM in Tris-buffered saline (TBS) containing 10% glycerol) in 96-well white flat bottom plates. The effect of molecular crowding was monitored by incubating the sensor and the protease in the absence or presence of 25% (w/v) of polyethylene glycol (PEG; 8000 Da) (Sigma-Aldrich). BRET measurements were performed at 37° C. for 3 h by the addition of furimazine (Promega, Wisconsin, USA) at a dilution of 1:200.

BRET and fluorescence measurements: BRET measurements were performed using a Tecan SPARK® multimode microplate reader. For live cell assays, bioluminescence spectral scan was performed from 380 to 664 nm wavelengths with an acquisition time of 400 ms for each wavelength to determine relative emissions from Nluc (donor) and mNG (acceptor) and quantify BRET, which is expressed as a ratio of emissions at 533 nm and 467 nm. Total mNG fluorescence in the sensor expressing cells were measured by exciting the samples at 480 nm and emission acquired at a wavelength of 530 nm.

### Results and Analysis

Data analysis and figure preparation: MD simulation data were plotted using Matplotlib in a Jupyter Notebook. GraphPad Prism (version 9 for MacOS, GraphPad Software, La Jolla California USA; www.graphpad.com) and Microsoft Excel will be used for data analysis and graph preparation. The results are presented as mean±SD obtained from a minimum of three experiments.

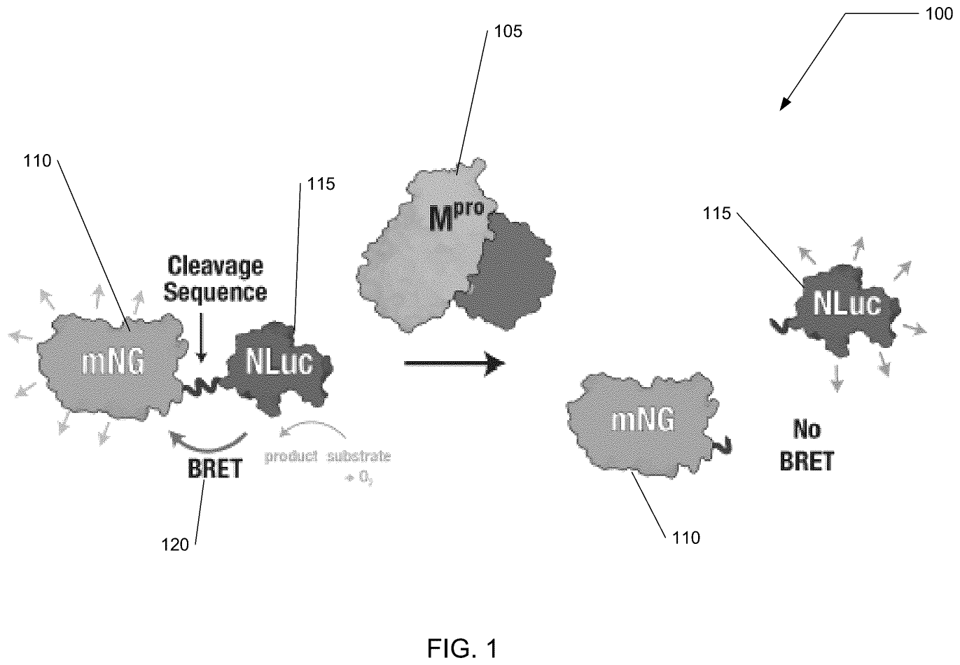

Designing BRET-based M^{pro} biosensors for increased cleavage rate: The Disclosed Invention is a highly specific, genetically encoded biosensor for monitoring SARS-COV-2 M^{pro} proteolytic activity in live cells using the BRET technology (FIG. 1). BRET is a phenomenon wherein energy from a luciferase protein (donor) is non-radiatively transferred through resonance energy transfer to a fluorescent protein (acceptor). BRET efficiency critically depends on the overlap of donor emission and acceptor excitation spectra. Additionally, it depends on the distance between the donor and acceptor (varies inversely with the 6th power of the distance), and thus, an increase in the distance between the two results in a large decrease in the BRET efficiency that can be detected as a change in the acceptor emission (FIG. 1). The Disclosed Invention utilizes the technique in developing a highly sensitive and specific biosensors for detecting intracellular levels of the second messenger cyclic guanosine monophosphate (cGMP) as well as detecting allosteric ligand binding-induced conformational changes in the multidomain Phosphodiesterase 5 (PDE5) protein. The Disclosed Invention generates a BRET-based M^{pro} proteolytic cleavage biosensor 100 by inserting the N-terminal autocleavage peptide sequence of M^{pro} (AVLQSGFR) 105 in between the mNeonGreen (mNG) 110 and NanoLuc (NLuc) proteins 115 (FIG. 1). The use of BRET 120, with NLuc as the bioluminescence donor and mNG as the resonance energy acceptor, allowed highly specific and sensitive monitoring of M^{pro} activity in live cells as well as in vitro.

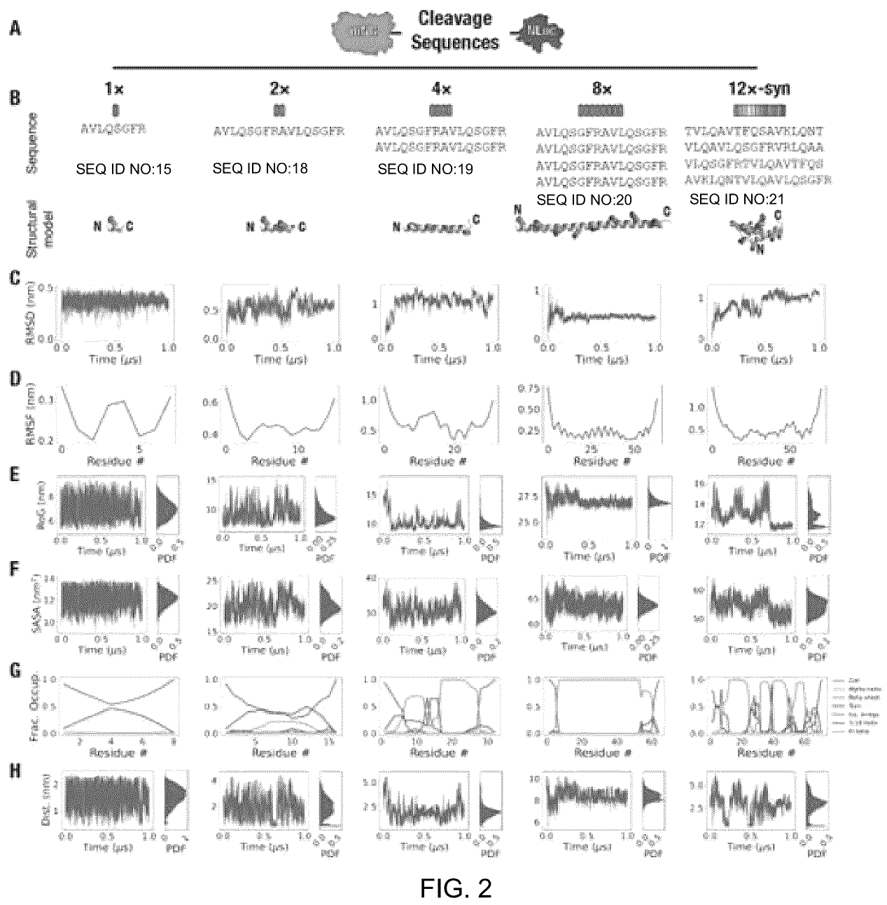

In order to engineer BRET-based M^{pro} biosensors with increased rate of cleavage compared to the originally designed biosensor containing a single M^{pro} N-terminal cleavage sequence, the disclosed invention includes a number of synthetic M^{pro} cleavage sequences. First, synthetic cleavage sequences containing either 2×, 4× and 8× repeats of the original Mpro N-terminal cleavage sequence were designed, which is expected to increase the cleavage rate by providing the indicated number of cleavage sequences per biosensor protein molecule (higher apparent concentration of the cleavage sequence; FIG. 2B).

Second, the disclosed invention includes a combination of M^{pro} cleavage sequences using the known M^{pro} cleavage sequences that contained a total of 12 cleavage sites while amino acid residues C-terminal to the critical Gln residue were varied (12×-syn-Mpro) (FIG. 2B). This maximizes the number of cleavage sites, which is expected to result in an increased rate of cleavage, while keeping the length of the synthetic construct to a minimal so that efficient BRET is observed in the intact state of the biosensor. For this, the secondary structures of the designed cleavage sequences were predicted and found to be predominantly adopting an alpha-helical structure, which is similar to the predicted structure of the original M^{pro} Nter cleavage sequence. Second, the three-dimensional model of the cleavage sequences were determined using the trRosetta algorithm, which combines the well-established Rosetta structure prediction pipeline with structural restraints determined from a trained deep neural network model of known structures. This revealed a long helical structure for the 2×, 4× and 8×-M^{pro}-Nter cleavage sequences while a significantly folded alpha-helical structure for the 12×-syn-M^{pro} cleavage sequence (FIG. 2B; lower panel).

To assess the structural dynamics and effective length (end-to-end distance) of the M^{pro} cleavage sequences, all atom, explicit solvent, GaMD simulations for each of the cleavage sequences were performed using NAMD (version 2.13) (FIG. 2). RMSD analysis of the backbone Ca atoms over the course of about 1 μs GaMD simulation revealed significant deviations in the structure of both the 4×-M^{pro}-Nter and 12×-M^{pro}-syn cleavage sequences, which was greater than that observed for the 1×-M^{pro}-Nter original cleavage sequence (FIG. 2C). RMSF analysis of the synthetic cleavage sequences, on the other hand, revealed a varied fluctuation of individual amino acid residues in them (FIG. 2D). Taken together, results from RMSD and RMSF analysis of the cleavage sequences revealed that they could serve as excellent substrates for M^{pro}. Further, the radius of gyration (RoG) was determined, which relates to the approximate size of the cleavage sequence, as well as end-to-end distances to assess the possibility of resonance energy transfer (RET) in BRET-based constructs wherein these cleavage sequences are sandwiched between mNG and NLuc, acceptor fluorescent and donor bioluminescent proteins respectively (FIG. 2E,F). Both RoG and end-to-end distance analysis of the cleavage sequence over the GaMD simulation trajectory indicate that while the synthetic cleavage sequences consists of many more amino acid residues, the effective distance between the N-and C-terminal residues in the synthetic M^{pro} cleavage sequences remain relatively lower (˜2 nm for 4×-M^{pro}-Nter and ˜4 nm for 12×-syn-M^{pro} as compared to ˜2 nm of the 1×-M^{pro}-Nter sequence, all of which are below the Forster's distance for resonance energy transfer between NLuc and mNG (˜5 nm), indicative of their utility in a BRET-based assay.

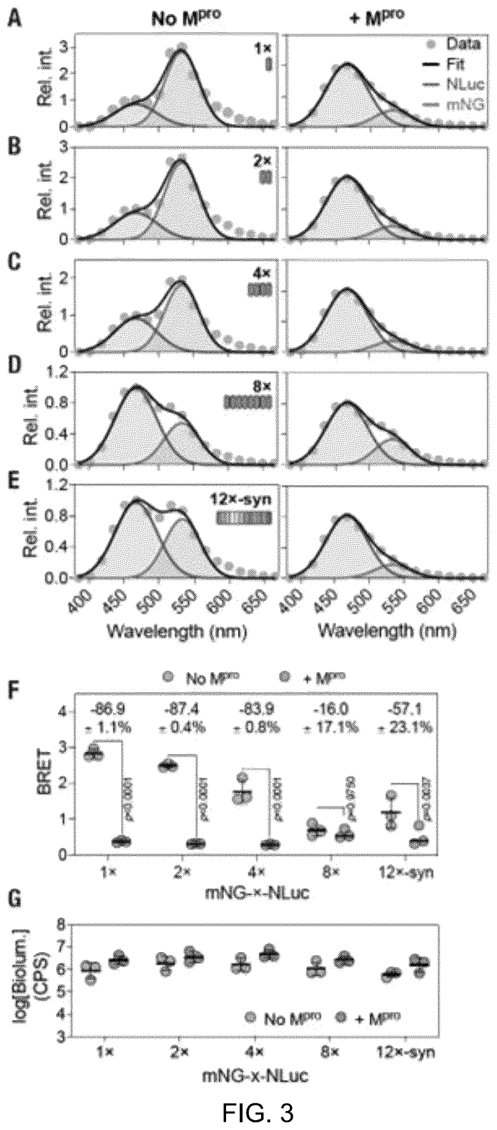

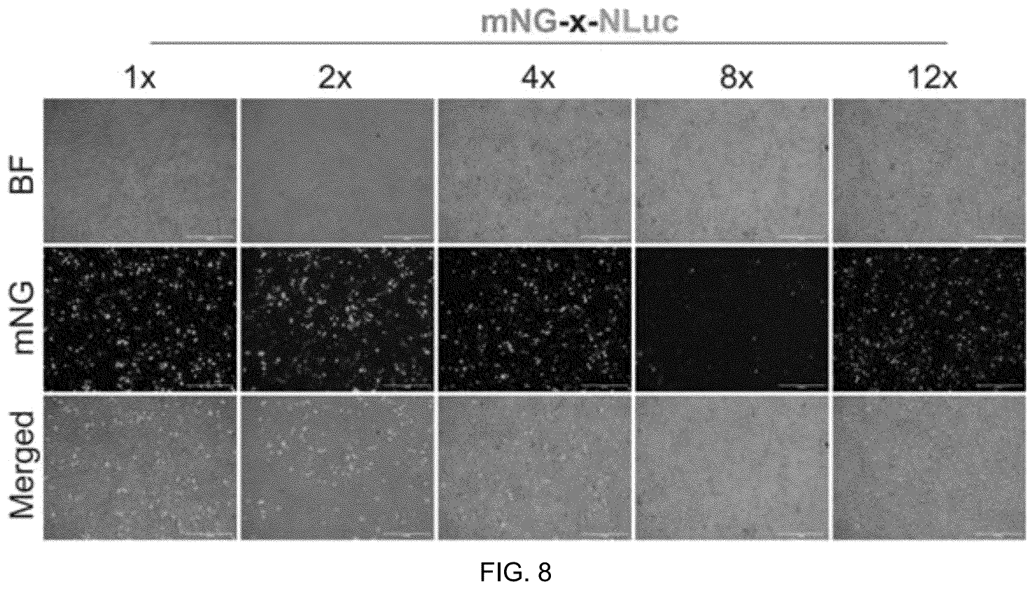

Characterization of M^{pro} biosensor constructs in live cells: To test the constructs for their ability to produce BRET signal and detect M^{pro}-mediated cleavage, the disclosed invention includes plasmid DNA constructs expressing 2×, 4×, 8× and 12×-syn M^{pro} cleavage sites (henceforth referred to as 2×, 4×, 8× and 12×-syn M^{pro} biosensors, respectively) and transfected them in HEK293T cells in addition to the 1× M^{pro} biosensor reported previously (FIG. 8). In FIG. 8, bright field (BF) and epifluorescence (mNG) images were acquired using a 10× objective of HEK293T cells transfected with various M^{pro} biosensors expressing plasmids showing robust expression of the biosensor constructs in these cells. Measurement of bioluminescence spectra in cells expressing various M^{pro} biosensor constructs showed two emission peaks characteristic of NLuc (467 nm) and mNG (533 nm) (FIG. 3A-E) and high BRET (FIG. 3F). Importantly, the efficiency of energy transfer between NLuc and mNG generally decreased with the increase in the length of cleavage sequences and the 12×-syn M^{pro} biosensor showed relatively high energy transfer given that the total length of the cleavage site was 68 amino acid residues likely reflecting the compact structure of the cleavage site obtained protein structure modeling and MD simulation (FIG. 2B,E,F). Additionally, expression of M^{pro} along with the biosensor constructs resulted in a large decrease in the mNG emission peak in all constructs, except for the 8× M^{pro} biosensor (FIG. 3A-E), leading to a decrease in BRET (FIG. 3F), indicating that the biosensors are cleaved by M^{pro} in the cells. Expression of the biosensors was monitored using total bioluminescence in cells expressing various M^{pro} biosensor constructs (FIG. 3G) and were found to be similar for all biosensor constructs.

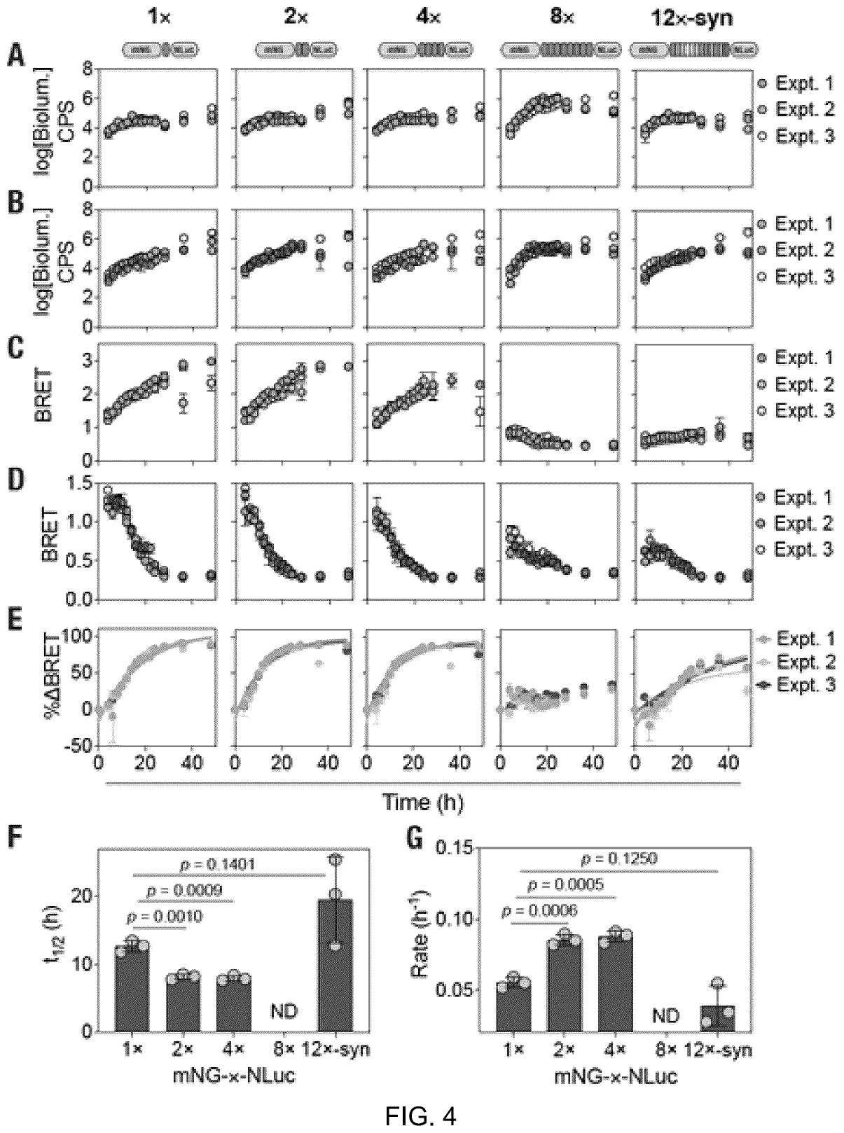

Increased rate of cleavage of the 2× and 4× M^{pro} biosensors in living cells: The temporal dynamics of M^{pro}-mediated cleavage of each biosensor construct in living cells were monitored. The cells were transfected with various M^{pro} biosensor constructs, either alone or along with M^{pro}, and BRET was determined at different time points (FIG. 4). Total bioluminescence activity over time showed the expression of the biosensor constructs in the absence (FIG. 4A) on in the presence (FIG. 4B) of M^{pro}, except for the 8× cleavage site containing biosensor, which a reduced expression in the initial time points but reached similar levels at later time points. The 1×, 2× and 4× M^{pro} biosensors showed a time-dependent increase in BRET reaching a plateau at ˜24 h post-transfection in the absence of M^{pro} expression (FIG. 4C). This likely reflects a relatively slower maturation of mNG as compared to NLuc in these biosensors, as reported previously. However, such increases in BRET were not observed for the 8× and 12×-syn M^{pro} biosensors (FIG. 4C), suggesting a reduced resonance energy transfer between NLuc and mNG in these biosensors. Importantly, expression of M^{pro} along with the biosensor constructs resulted in a discernable decrease in BRET as early as 8 h post-transfection and continued to decrease at later time points, except for the 8× M^{pro} biosensor, (FIG. 4D), indicating a delayed but a robust M^{pro}-mediated cleavage of the biosensor constructs.

To assess the rate of cleavage of the biosensors, the percentage change in BRET in the presence of M^{pro}, from that in the absence of M^{pro}, over time was determined. This analysis revealed a faster cleavage kinetics for the 2× and 4× M^{pro} biosensors (FIG. 4E). On the other hand, the 8× and 12×-syn M^{pro} biosensors showed no or slower cleavage kinetics, respectively (FIG. 4E). For a quantitative comparison, percentage change in BRET, i.e. cleavage kinetics, data was fitted to a one phase association model and determined the cleavage half-time (t_{1/2}) and rate. This revealed that the 2× and 4× M^{pro} biosensors exhibited shorter t1/2 (8.1±0.4 h and 7.9±0.4 h, respectively), and a higher cleavage rate (0.085±0.004 h−1 and 0.088±0.004 h−1, respectively) as compared to 1× M^{pro} biosensor (12.6±0.8 h and 0.055±0.004 h−1, respectively) (FIG. 4F,G) (i.e. the 2× and 4× M^{pro} biosensors showed ˜1.6 times faster cleavage rate, as compared to the 1× M^{pro} biosensor). In contrast, the 12×-syn M^{pro} biosensor showed a longer t_{1/2} (19.5±6.4 h) and a slower cleavage rate (0.039±0.001 h−1) (FIG. 4F,G). Percentage change in BRET data for the 8× M^{pro} biosensor was not fit to the model due to a lack of change in BRET. These results suggest that the 2× and 4× M^{pro} biosensors served as a better substrate for proteolytic cleavage by M^{pro}. The 2× M^{pro} biosensor was utilized due its smaller size and a higher BRET in the absence of M^{pro}, as compared to the 4× M^{pro} biosensor (2.548±0.426 vs. 2.053±0.504).

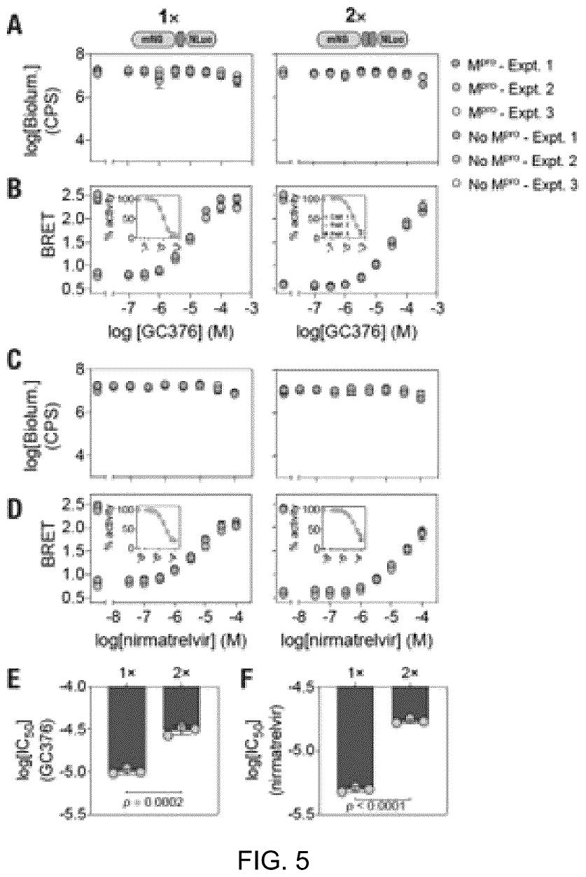

Decreased M^{pro} inhibition efficacy of pharmacological inhibitors in cells expressing the 2× M^{pro} biosensor: Cells expressing either the 1× or the 2× M^{pro} biosensors were treated with a range of concentrations of the covalent inhibitor, GC376, and non-covalent inhibitor, nirmatrelvir. Total bioluminescence measurements revealed similar expression of the biosensors (FIG. 5A) while BRET measurements after 16 h of GC376 treatment revealed a concentration-dependent decrease in the M^{pro} proteolytic cleavage activity, as determined from increases in BRET, although the inhibition did not reach saturation in the case of the 2× M^{pro} biosensor (FIG. 5B). Similar results were obtained for the total bioluminescence (FIG. 5C) and inhibition of M^{pro} proteolytic cleavage activity (FIG. 5D) with nirmatrelvir. Fitting of the percentage remaining M^{pro} proteolytic cleavage activity revealed GC376 IC50 values of 10.06±0.75 and ˜30.43±3.53 uM and nirmatrelvir IC50 values of 4.93±0.22 and ˜16.96±0.66 μM for the 1× and 2× M^{pro} biosensors, respectively. Thus, GC376 showed a ˜3-fold and nirmatrelvir showed a ˜3.5-fold decrease in their efficacy in inhibiting M^{pro} in the presence of the 2× M^{pro} biosensor. The decrease in the efficacy of the pharmacological inhibitors in the presence of the 2× Mpro biosensor observed here could result from its faster cleavage rate, and the difference in the extent of decrease of efficacy of the inhibitors likely reflects their nature of interaction with M^{pro} (i.e. covalent vs. non-covalent binding of GC376 and nirmatrelvir, respectively.

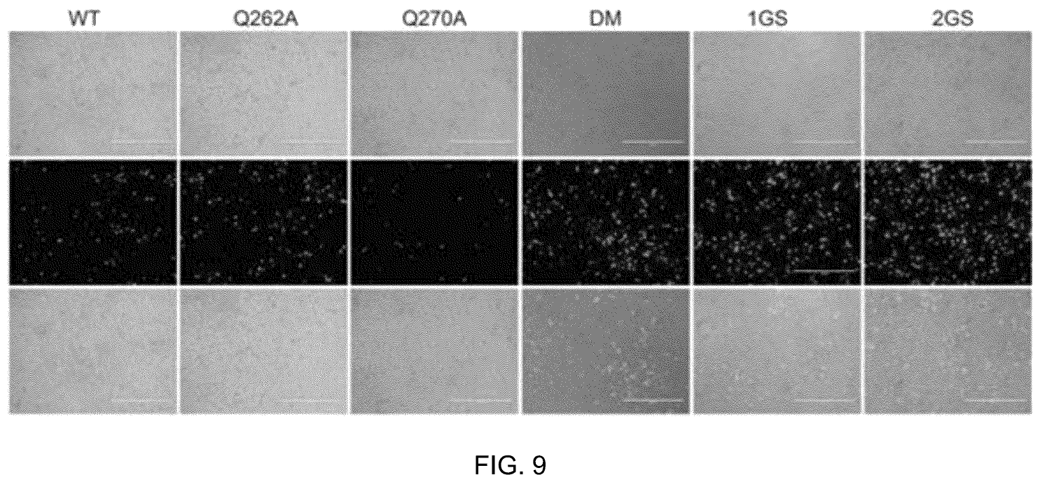

Mutational analysis reveals a requirement for both cleavage sites for the increased cleavage rate of the 2× M^{pro} biosensor: The underlying molecular requirements and basis for the observations made with the disclosed invention were determined. For this, first the critical Gln residue was mutated to an Ala residue (from AVLQSGFR to AVLASGFR) either in the first (Q262A) or the second (Q270A) or both (DM) cleavage sequences. Second, the first (1GS) or the second (2GS) cleavage sequence were replaced with a Gly-Ser linker of the same length (from AVLQSGFR to GSGSGSGS). Then HEK293T cells were transfected with the wild type (WT) and the mutant 2×, along with the original 1×, M^{pro} biosensor, expressing plasmids, either in the absence or in the presence of M^{pro}. FIG. 9 shows right field (top panel) and epifluorescence (bottom) images acquired using a 10× objective of HEK 293T cells transfected with various 2× M^{pro} biosensor expressing plasmids showing robust expression of the biosensors in these cells.

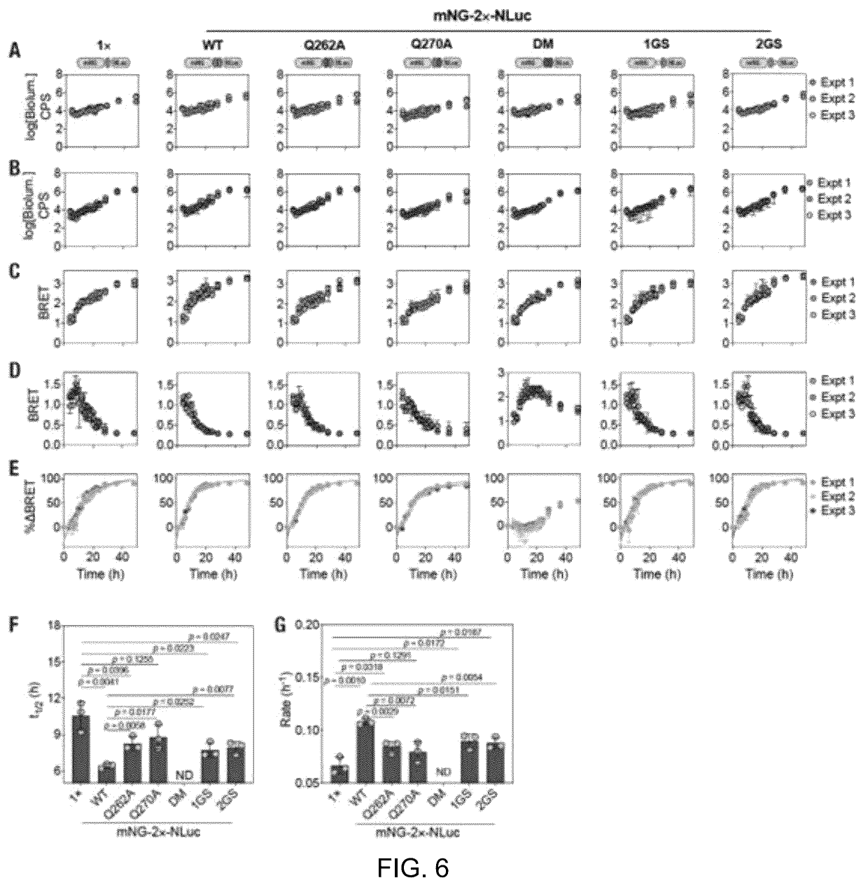

Total bioluminescence measurements revealed similar trends in the expression of all the biosensors, either in the absence (FIG. 6A) or in the presence (FIG. 6B) of M^{pro}. BRET measurements revealed a similar trend of increase in BRET over time in the absence of M^{pro} (FIG. 6C) and a decrease in BRET in the presence of M^{pro}, except for the DM 2× M^{pro} biosensor that showed an initial increase and then decrease in BRET (FIG. 6D). The percentage change in BRET from those in the absence to those in the presence of M^{pro} over time (FIG. 6E) was determined, and the data was fitted to a one-phase association model, as done above, to determine the t_{1/2} and cleavage rate of each biosensor (FIG. 6F,G). This analysis revealed that both Q262A (in the first cleavage site) and Q270A (in the second cleavage site) mutation in the 2× M^{pro} biosensor resulted in an increase in the t_{1/2} (8.255±0.560 and 8.786±1.037 h of the Q262A and Q270A mutant, respectively, vs. 6.420±0.186 h of the WT) and a decrease in the cleavage rate (0.084±0.005 and 0.084±0.005 h⁻¹ of the 1GS and 2GS mutant, respectively, vs. 0.108±0.003 h⁻¹ of the WT), as compared to the WT 2× M^{pro} biosensor, while the DM (Q262A/Q270A) resulted in a complete loss of M^{pro}-mediated cleavage (FIG. 6F,G).

Similarly, both the 1GS as well as the 2GS mutations in the 2× M^{pro} biosensor resulted in an increase in the t_{1/2} (7.727±0.622 and 7.928±0.492 h of the 1GS and 2GS mutant, respectively, vs. 6.420±0.186 h of the WT) and a decrease in the cleavage rate (0.090±0.007and 0.088±0.006 h⁻¹ of the 1GS and 2GS mutant, respectively, vs. 0.108±0.003 h⁻¹ of the WT), as compared to the WT 2× M^{pro} biosensor (FIG. 6F,G). While the Q262A, Q270A, 1GS and 2GS mutant 2× M^{pro} biosensors showed an increased t_{1/2} and a decrease cleavage rate in comparison to the WT 2× M^{pro} biosensor, these mutants still showed a shorter t_{1/2} and an increased cleavage rate in comparison to the 1× M^{pro} biosensor (FIG. 6F,G). Together, these results suggest that while a longer linker (16 residues) containing one or the other cleavage site mutated between mNG and NLuc might show a better M^{pro}-mediated cleavage rate in comparison 1× M^{pro} biosensor (8 residues), both cleavage sites are required for the much-enhanced cleavage rate of the 2× M^{pro} biosensor.

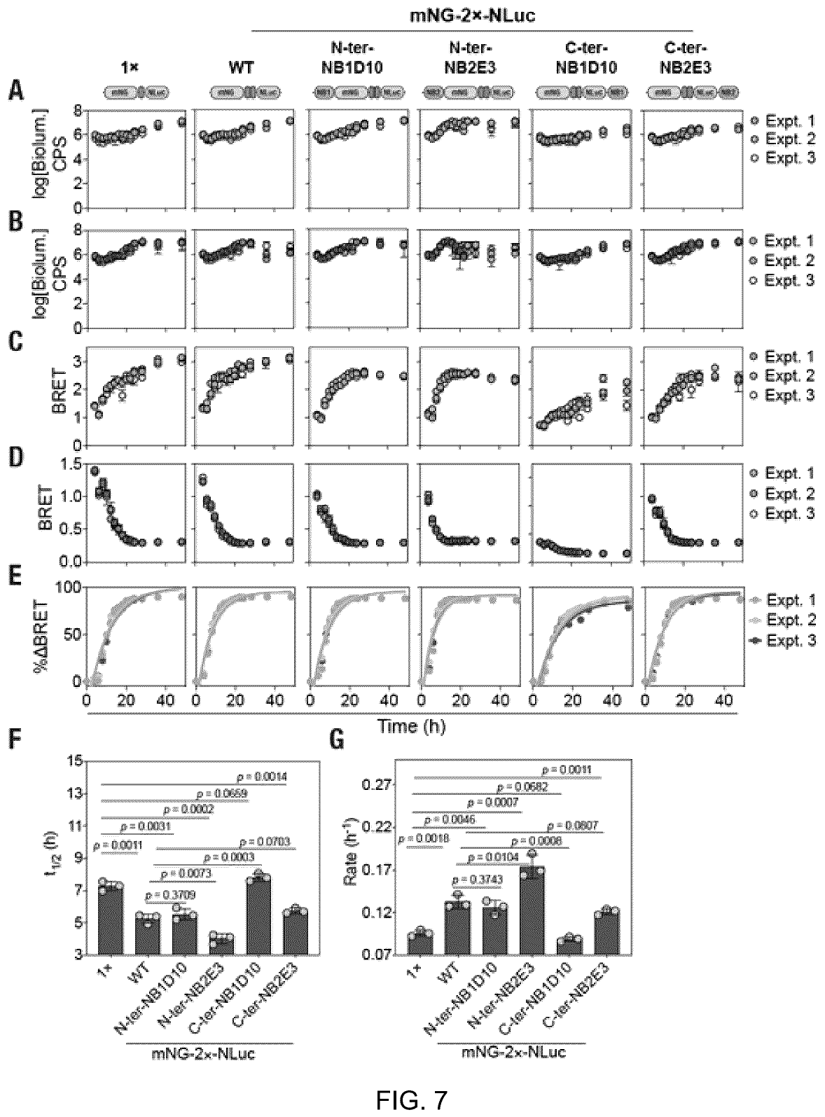

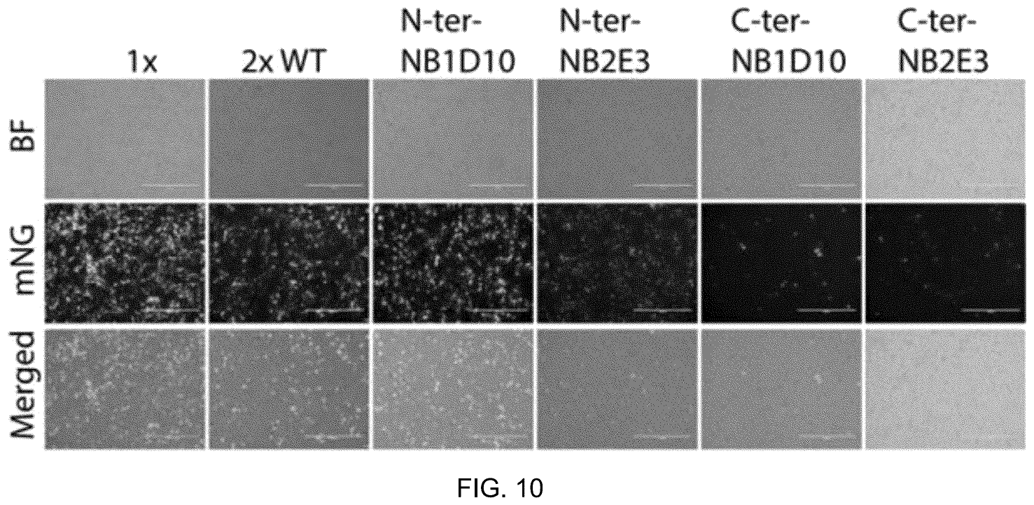

N-terminal NB2E3 nanobody containing 2× M^{pro} biosensor shows further increased cleavage rate: To further increase the cleavage rate of the 2× M^{pro} biosensor and thus, increase the sensitivity of M^{pro}-mediated cleavage of the biosensor, the local concentration of M^{pro} was increased through the fusion of M^{pro}-binding nanobodies to the 2× M^{pro} biosensor. Nanobodies are single-domain antibody fragments derived from the variable region of the heavy chain and are characterized by their efficient antigen-binding property, in addition to their small size (12-15 kD), long shelf-life, high stability, and solubility. In this regard, nanobodies against M^{pro} that can bind and inhibit M^{pro} activity as well as those that bind M^{pro}, but do not inhibit its activity were used. Specifically, the nanobodies NB1D10 and NB2E3 were chosen and generated both N-terminal (N-ter-NB1D10 and N-ter-NB2E3) as well as C-terminal (C-ter-NB1D10 and C-ter-NB2E3) fusions of the nanobodies with the 2× M^{pro} biosensor and transfected cells these nanobody-containing 2× M^{pro} biosensors, along with the 2× and 1× M^{pro} biosensors, either in the absence (FIG. 10) or in the presence of M^{pro}. In FIG. 10, epifluorescence images acquired using a 10× objective of HEK 293T cells transfected with plasmids of pmNG-2×-NLuc containing nanobody showing robust expression of the biosensor constructs in these cells. Total bioluminescence measurements revealed similar expression of the biosensors either in the absence (FIG. 7A) or in the presence (FIG. 7B) of M^{pro}. BRET measurements revealed a time-dependent increase, with reaching a plateau after 24 h post-transfection, in BRET in the absence of M^{pro} (FIG. 7C) and a decrease in the presence of M^{pro} (FIG. 7D) of all biosensors, except the C-ter NB1D10 2× M^{pro} biosensor.

As previously noted, time-dependent, percentage change in BRET was determined (FIG. 7E). The t_{1/2} and cleavage rate of each these biosensors was also determined through fitting the data to a one-phase association model (FIG. 7F,G). This analysis revealed a differential effect of nanobody fusions to the 2× M^{pro} biosensor in that the N-ter-NB1D10 and C-ter-NB2E3 2× M^{pro} biosensor showed similar t_{1/2} (5.527±0.371 and 5.735±0.165 h of the N-ter-NB1D10 and C-ter-NB2E3, respectively, vs. 5.249±0.301 h of the WT 2× M^{pro} biosensor) and cleavage rates (0.126±0.008 and 0.121±0.003 h⁻¹ of the N-ter-NB1D10 and C-ter-NB2E3, respectively, vs. 0.132±0.008 h⁻¹ of the WT 2× M^{pro} biosensor) (FIG. 7F,G). On the other hand, the N-ter-NB2E3 2× M^{pro} biosensor showed a decreased t_{1/2} (4.004±0.305 h) and an increased cleavage rate (0.174±0.014h⁻¹) while the C-ter-NB1D10 2× M^{pro} biosensor showed an increased t_{1/2} (7.811±0.241 h) and a decreased cleavage rate (0.089±0.003 h⁻¹), as compared to the WT 2× M^{pro} biosensor (FIG. 7F,G). All 2× M^{pro} biosensor, except the C-ter-NB1D10, showed reduced t_{1/2} and increased cleavage rate in comparison to the 1× M^{pro} biosensor (FIG. 7F,G). Overall, these results suggest that the N-ter-NB2E3 2× M^{pro} biosensor showed the fastest cleavage rate, likely due to recruitment of M^{pro} to the biosensor in a way that results in an efficient cleavage of the 2× cleavage. In contrast, the C-terminal fusion of the NB1D10 nanobody to the 2× M^{pro} biosensor resulted in a decrease in the cleavage rate, compared to the 2× M^{pro} biosensor without any nanobody likely due to recruitment of M^{pro} to the biosensor in a way that results in reduction in the cleavage efficiency.

TABLE 1
M^{pro} biosensor construct sequences

| SEQ ID | | |
|---|---|---|
| NO | CONSTRUCT | |
| 1 | mNG-M^{pro}-1×- | MGSSHHHHHHSSGLVPRGSHMVSKGEEDNMASLPATHELHIFGSINGVDFDMVGQG |
| | NLuc | TGNPNDGYEELNLKSTKGDLQFSPWILVPHIGYGFHQYLPYPDGMSPFQAAMVDGS |
| | | GYQVHRTMQFEDGASLTVNYRYTYEGSHIKGEAQVKGTGFPADGPVMTNSLTAADW |
| | | CRSKKTYPNDKTIISTFKWSYTTGNGKRYRSTARTTYTFAKPMAANYLKNQPMYVF |
| | | RKTELKHSKTELNFKEWQKAFTDVMGMDELYKEFGTENLYAVLQSGFRGSGGSMVF |
| | | TLEDFVGDWRQTAGYNLDQVLEQGGVSSLFQNLGVSVTPIQRIVLSGENGLKIDIH |
| | | VIIPYEGLSGDQMGQIEKIFKVVYPVDDHHFKVILHYGTLVIDGVTPNMIDYFGRP |
| | | YEGIAVFDGKKITVTGTLWNGNKIIDERLINPDGSLLFRVTINGVTGWRLCERILA |
| 2 | mNG-M^{pro}-2×- | MGSSHHHHHHSSGLVPRGSHMVSKGEEDNMASLPATHELHIFGSINGVDFDMVGQG |
| | NLuc | TGNPNDGYEELNLKSTKGDLQFSPWILVPHIGYGFHQYLPYPDGMSPFQAAMVDGS |
| | | GYQVHRTMQFEDGASLTVNYRYTYEGSHIKGEAQVKGTGFPADGPVMTNSLTAADW |
| | | CRSKKTYPNDKTIISTFKWSYTTGNGKRYRSTARTTYTFAKPMAANYLKNQPMYVF |
| | | RKTELKHSKTELNFKEWQKAFTDVMGMDELYKGTAVLQSGFRAVLQSGFRAAAMVF |
| | | TLEDFVGDWRQTAGYNLDQVLEQGGVSSLFQNLGVSVTPIQRIVLSGENGLKIDIH |
| | | VIIPYEGLSGDQMGQIEKIFKVVYPVDDHHFKVILHYGTLVIDGVTPNMIDYFGRP |
| | | YEGIAVFDGKKITVTGTLWNGNKIIDERLINPDGSLLFRVTINGVTGWRLCERILA |
| 3 | mNG-M^{pro}-4×- | MGSSHHHHHHSSGLVPRGSHMVSKGEEDNMASLPATHELHIFGSINGVDFDMVGQG |
| | NLuc | TGNPNDGYEELNLKSTKGDLQFSPWILVPHIGYGFHQYLPYPDGMSPFQAAMVDGS |
| | | GYQVHRTMQFEDGASLTVNYRYTYEGSHIKGEAQVKGTGFPADGPVMTNSLTAADW |
| | | CRSKKTYPNDKTIISTFKWSYTTGNGKRYRSTARTTYTFAKPMAANYLKNQPMYVF |
| | | RKTELKHSKTELNFKEWQKAFTDVMGMDELYKGTAVLQSGFRAVLQSGFRAVLQSG |
| | | FRAVLQSGFRAAAMVFTLEDFVGDWRQTAGYNLDQVLEQGGVSSLFQNLGVSVTPI |
| | | QRIVLSGENGLKIDIHVIIPYEGLSGDQMGQIEKIFKVVYPVDDHHFKVILHYGTL |
| | | VIDGVTPNMIDYFGRPYEGIAVFDGKKITVTGTLWNGNKIIDERLINPDGSLLFRV |
| | | TINGVTGWRLCERILA |
| 4 | mNG-M^{pro}-8×- | MGSSHHHHHHSSGLVPRGSHMVSKGEEDNMASLPATHELHIFGSINGVDFDMVGQG |
| | NLuc | TGNPNDGYEELNLKSTKGDLQFSPWILVPHIGYGFHQYLPYPDGMSPFQAAMVDGS |
| | | GYQVHRTMQFEDGASLTVNYRYTYEGSHIKGEAQVKGTGFPADGPVMTNSLTAADW |
| | | CRSKKTYPNDKTIISTFKWSYTTGNGKRYRSTARTTYTFAKPMAANYLKNQPMYVF |
| | | RKTELKHSKTELNFKEWQKAFTDVMGMDELYKGTAVLQSGFRAVLQSGFRAVLQSG |
| | | FRAVLQSGFRAVLQSGFRAVLQSGFRAVLQSGFRAVLQSGFRAAAMVFTLEDFVGD |
| | | WRQTAGYNLDQVLEQGGVSSLFQNLGVSVTPIQRIVLSGENGLKIDIHVIIPYEGL |
| | | SGDQMGQIEKIFKVVYPVDDHHFKVILHYGTLVIDGVTPNMIDYFGRPYEGIAVFD |
| | | GKKITVTGTLWNGNKIIDERLINPDGSLLFRVTINGVTGWRLCERILA |
| 5 | mNG-M^{pro}-12×- | MGSSHHHHHHSSGLVPRGSHMVSKGEEDNMASLPATHELHIFGSINGVDFDMVGQG |
| | syn-NLuc | TGNPNDGYEELNLKSTKGDLQFSPWILVPHIGYGFHQYLPYPDGMSPFQAAMVDGS |
| | | GYQVHRTMQFEDGASLTVNYRYTYEGSHIKGEAQVKGTGFPADGPVMTNSLTAADW |
| | | CRSKKTYPNDKTIISTFKWSYTTGNGKRYRSTARTTYTFAKPMAANYLKNQPMYVF |
| | | RKTELKHSKTELNFKEWQKAFTDVMGMDELYKGTTVLQAVTFQSAVKLQNTVLQAV |
| | | LQSGFRVRLQAAVLQSGFRTVLQAVTFQSAVKLQNTVLQAVLQSGFRAAAMVFTLE |
| | | DFVGDWRQTAGYNLDQVLEQGGVSSLFQNLGVSVTPIQRIVLSGENGLKIDIHVII |
| | | PYEGLSGDQMGQIEKIFKVVYPVDDHHFKVILHYGTLVIDGVTPNMIDYFGRPYEG |
| | | IAVFDGKKITVTGTLWNGNKIIDERLINPDGSLLFRVTINGVTGWRLCERILA |
| 6 | mNG-M^{pro}-2× | MGSSHHHHHHSSGLVPRGSHMVSKGEEDNMASLPATHELHIFGSINGVDFDMVGQG |
| | (Q262A)-NLuc | TGNPNDGYEELNLKSTKGDLQFSPWILVPHIGYGFHQYLPYPDGMSPFQAAMVDGS |
| | | GYQVHRTMQFEDGASLTVNYRYTYEGSHIKGEAQVKGTGFPADGPVMTNSLTAADW |
| | | CRSKKTYPNDKTIISTFKWSYTTGNGKRYRSTARTTYTFAKPMAANYLKNQPMYVF |
| | | RKTELKHSKTELNFKEWQKAFTDVMGMDELYKGTAVLASGFRAVLQSGFRAAAMVF |
| | | TLEDFVGDWRQTAGYNLDQVLEQGGVSSLFQNLGVSVTPIQRIVLSGENGLKIDIH |
| | | VIIPYEGLSGDQMGQIEKIFKVVYPVDDHHFKVILHYGTLVIDGVTPNMIDYFGRP |
| | | YEGIAVFDGKKITVTGTLWNGNKIIDERLINPDGSLLFRVTINGVTGWRLCERILA |
| 7 | mNG-M^{pro}-2× | MGSSHHHHHHSSGLVPRGSHMVSKGEEDNMASLPATHELHIFGSINGVDFDMVGQG |
| | (Q270A)-NLuc | TGNPNDGYEELNLKSTKGDLQFSPWILVPHIGYGFHQYLPYPDGMSPFQAAMVDGS |
| | | GYQVHRTMQFEDGASLTVNYRYTYEGSHIKGEAQVKGTGFPADGPVMTNSLTAADW |
| | | CRSKKTYPNDKTIISTFKWSYTTGNGKRYRSTARTTYTFAKPMAANYLKNQPMYVF |
| | | RKTELKHSKTELNFKEWQKAFTDVMGMDELYKGTAVLQSGFRAVLASGFRAAAMVF |
| | | TLEDFVGDWRQTAGYNLDQVLEQGGVSSLFQNLGVSVTPIQRIVLSGENGLKIDIH |
| | | VIIPYEGLSGDQMGQIEKIFKVVYPVDDHHFKVILHYGTLVIDGVTPNMIDYFGRP |
| | | YEGIAVFDGKKITVTGTLWNGNKIIDERLINPDGSLLFRVTINGVTGWRLCERILA |
| 8 | mNG-M^{pro}-2× | MGSSHHHHHHSSGLVPRGSHMVSKGEEDNMASLPATHELHIFGSINGVDFDMVGQG |
| | (Q262A/ | TGNPNDGYEELNLKSTKGDLQFSPWILVPHIGYGFHQYLPYPDGMSPFQAAMVDGS |
| | Q270A)-NLuc | GYQVHRTMQFEDGASLTVNYRYTYEGSHIKGEAQVKGTGFPADGPVMTNSLTAADW |
| | | CRSKKTYPNDKTIISTFKWSYTTGNGKRYRSTARTTYTFAKPMAANYLKNQPMYVF |
| | | RKTELKHSKTELNFKEWQKAFTDVMGMDELYKGTAVLASGFRAVLASGFRAAAMVF |
| | | TLEDFVGDWRQTAGYNLDQVLEQGGVSSLFQNLGVSVTPIQRIVLSGENGLKIDIH |
| | | VIIPYEGLSGDQMGQIEKIFKVVYPVDDHHFKVILHYGTLVIDGVTPNMIDYFGRP |
| | | YEGIAVFDGKKITVTGTLWNGNKIIDERLINPDGSLLFRVTINGVTGWRLCERILA |
| 9 | mNG-M^{pro}-2×- | MGSSHHHHHHSSGLVPRGSHMVSKGEEDNMASLPATHELHIFGSINGVDFDMVGQG |
| | 1GS-NLuc | TGNPNDGYEELNLKSTKGDLQFSPWILVPHIGYGFHQYLPYPDGMSPFQAAMVDGS |
| | | GYQVHRTMQFEDGASLTVNYRYTYEGSHIKGEAQVKGTGFPADGPVMTNSLTAADW |
| | | CRSKKTYPNDKTIISTFKWSYTTGNGKRYRSTARTTYTFAKPMAANYLKNQPMYVF |
| | | RKTELKHSKTELNFKEWQKAFTDVMGMDELYKGTGSGSGSGSAVLQSGFRAAAMVF |
| | | TLEDFVGDWRQTAGYNLDQVLEQGGVSSLFQNLGVSVTPIQRIVLSGENGLKIDIH |
| | | VIIPYEGLSGDQMGQIEKIFKVVYPVDDHHFKVILHYGTLVIDGVTPNMIDYFGRP |
| | | YEGIAVFDGKKITVTGTLWNGNKIIDERLINPDGSLLFRVTINGVTGWRLCERILA |
| 10 | mNG-M^{pro}-2×- | MGSSHHHHHHSSGLVPRGSHMVSKGEEDNMASLPATHELHIFGSINGVDFDMVGQG |
| | 2GS-NLuc | TGNPNDGYEELNLKSTKGDLQFSPWILVPHIGYGFHQYLPYPDGMSPFQAAMVDGS |
| | | GYQVHRTMQFEDGASLTVNYRYTYEGSHIKGEAQVKGTGFPADGPVMTNSLTAADW |
| | | CRSKKTYPNDKTIISTFKWSYTTGNGKRYRSTARTTYTFAKPMAANYLKNQPMYVF |
| | | RKTELKHSKTELNFKEWQKAFTDVMGMDELYKGTAVLQSGFRGSGSGSGSAAAMVF |
| | | TLEDFVGDWRQTAGYNLDQVLEQGGVSSLFQNLGVSVTPIQRIVLSGENGLKIDIH |
| | | VIIPYEGLSGDQMGQIEKIFKVVYPVDDHHFKVILHYGTLVIDGVTPNMIDYFGRP |
| | | YEGIAVFDGKKITVTGTLWNGNKIIDERLINPDGSLLFRVTINGVTGWRLCERILA |
| 11 | NB1D10-mNG- | MGSSHHHHHHHHHHSSGLVPRGSHGSGSGSQVQLQESGGGSVQAGGSLRLSCAVSG |
| | M^{pro}-2×-NLuc | YSYCNYDLGWFRQAPGKEREGIATIDSDGNTYYVDSVKGRFTISQDNAKNTVALQM |
| | | NSLKPEDTAMYYCKVGSIASSVPEVSCPPSAPFGYWGQGTQVTVSSGSGSGSGSGS |
| | | GSGSGSGSGSGSGSMVSKGEEDNMASLPATHELHIFGSINGVDFDMVGQGTGNPND |
| | | GYEELNLKSTKGDLQFSPWILVPHIGYGFHQYLPYPDGMSPFQAAMVDGSGYQVHR |
| | | TMQFEDGASLTVNYRYTYEGSHIKGEAQVKGTGFPADGPVMTNSLTAADWCRSKKT |
| | | YPNDKTIISTFKWSYTTGNGKRYRSTARTTYTFAKPMAANYLKNQPMYVFRKTELK |
| | | HSKTELNFKEWQKAFTDVMGMDELYKGTAVLQSGFRAVLQSGFRAAAMVFTLEDFV |
| | | GDWRQTAGYNLDQVLEQGGVSSLFQNLGVSVTPIQRIVLSGENGLKIDIHVIIPYE |
| | | GLSGDQMGQIEKIFKVVYPVDDHHFKVILHYGTLVIDGVTPNMIDYFGRPYEGIAV |
| | | FDGKKITVTGTLWNGNKIIDERLINPDGSLLFRVTINGVTGWRLCERILA |
| 12 | NB2E3-mNG- | MGSSHHHHHHHHHHSSGLVPRGSHGSGSGSQVQLQESGGGSVQAGGSLRLSCAVSG |
| | M^{pro}-2×-NLuc | FIATSCALGWFRQAPGKEREGIATITTDGTTYYVDSVKGRFTISQDNAKNTVALQM |
| | | NSLKPEDTAMYYCKLCCSGQYCAWGQGTQVTVSSGSGSGSGSGSGSGSGSGSGSGS |
| | | GSMVSKGEEDNMASLPATHELHIFGSINGVDFDMVGQGTGNPNDGYEELNLKSTKG |
| | | DLQFSPWILVPHIGYGFHQYLPYPDGMSPFQAAMVDGSGYQVHRTMQFEDGASLTV |
| | | NYRYTYEGSHIKGEAQVKGTGFPADGPVMTNSLTAADWCRSKKTYPNDKTIISTFK |
| | | WSYTTGNGKRYRSTARTTYTFAKPMAANYLKNOPMYVFRKTELKHSKTELNFKEWQ |
| | | KAFTDVMGMDELYKGTAVLQSGFRAVLQSGFRAAAMVFTLEDFVGDWRQTAGYNLD |
| | | QVLEQGGVSSLFQNLGVSVTPIQRIVLSGENGLKIDIHVIIPYEGLSGDQMGQIEK |
| | | IFKVVYPVDDHHFKVILHYGTLVIDGVTPNMIDYFGRPYEGIAVFDGKKITVTGTL |
| | | WNGNKIIDERLINPDGSLLFRVTINGVTGWRLCERILA |
| 13 | mNG-M^{pro}-2×- | MGSSHHHHHHSSGLVPRGSHMVSKGEEDNMASLPATHELHIFGSINGVDFDMVGQG |
| | NLuc-NB1D10 | TGNPNDGYEELNLKSTKGDLQFSPWILVPHIGYGFHQYLPYPDGMSPFQAAMVDGS |
| | | GYQVHRTMQFEDGASLTVNYRYTYEGSHIKGEAQVKGTGFPADGPVMTNSLTAADW |
| | | CRSKKTYPNDKTIISTFKWSYTTGNGKRYRSTARTTYTFAKPMAANYLKNQPMYVF |
| | | RKTELKHSKTELNFKEWQKAFTDVMGMDELYKGTAVLQSGFRAVLQSGFRAAAMVF |
| | | TLEDFVGDWRQTAGYNLDQVLEQGGVSSLFQNLGVSVTPIQRIVLSGENGLKIDIH |
| | | VIIPYEGLSGDQMGQIEKIFKVVYPVDDHHFKVILHYGTLVIDGVTPNMIDYFGRP |
| | | YEGIAVFDGKKITVTGTLWNGNKIIDERLINPDGSLLFRVTINGVTGWRLCERILA |
| | | GSGSGSGSGSGSGSGSGSGSGSGSQVQLQESGGGSVQAGGSLRLSCAVSGYSYCNY |
| | | DLGWFRQAPGKEREGIATIDSDGNTYYVDSVKGRFTISQDNAKNTVALQMNSLKPE |
| | | DTAMYYCKVGSIASSVPEVSCPPSAPFGYWGQGTQVTVSS |
| 14 | mNG-M^{pro}-2×- | MGSSHHHHHHSSGLVPRGSHMVSKGEEDNMASLPATHELHIFGSINGVDFDMVGQG |
| | NLuc-NB2E3 | TGNPNDGYEELNLKSTKGDLQFSPWILVPHIGYGFHQYLPYPDGMSPFQAAMVDGS |
| | | GYQVHRTMQFEDGASLTVNYRYTYEGSHIKGEAQVKGTGFPADGPVMTNSLTAADW |
| | | CRSKKTYPNDKTIISTFKWSYTTGNGKRYRSTARTTYTFAKPMAANYLKNQPMYVF |
| | | RKTELKHSKTELNFKEWQKAFTDVMGMDELYKGTAVLQSGFRAVLQSGFRAAAMVF |
| | | TLEDFVGDWRQTAGYNLDQVLEQGGVSSLFQNLGVSVTPIQRIVLSGENGLKIDIH |
| | | VIIPYEGLSGDQMGQIEKIFKVVYPVDDHHFKVILHYGTLVIDGVTPNMIDYFGRP |
| | | YEGIAVFDGKKITVTGTLWNGNKIIDERLINPDGSLLFRVTINGVTGWRLCERILA |
| | | GSGSGSGSGSGSGSGSGSGSGSGSQVQLQESGGGSVQAGGSLRLSCAVSGFIATSC |
| | | ALGWFRQAPGKEREGIATITTDGTTYYVDSVKGRFTISQDNAKNTVALQMNSLKPE |
| | | DTAMYYCKLCCSGQYCAWGQGTQVTVSS |

Overall: 2×, 4×, 8× and 12×-syn M^{pro} cleavage sequences were designed for use in the development of a BRET-based M^{pro} biosensors increased cleavage rate compared to the 1× M^{pro} cleavage sequence. Following computational analysis, the biosensors were tested in live cells, both in terms of their BRET signal and cleavage efficiency, and it was found the 2× M^{pro} biosensor shows a high BRET, an increased cleavage rate and a decreased pharmacological inhibitor efficacy. Mutational analysis of the 2× M^{pro} biosensor showed a requirement for cleavage sites to be intact. Further, fusion of M^{pro} binding nanobodies to the 2× M^{pro} biosensor revealed the N-ter-NB2E3 2× M^{pro} biosensor to be the fastest cleaving M^{pro} biosensor. The N-ter-NB2E3 2× Mpro biosensor in the disclosed invention has application in the discovery of more potent pharmacological inhibitors of M^{pro} for COVID-19 treatment as well as in functional genomics applications such as characterizing mutations in the M^{pro} proteins that are observed in newly emerging SARS-COV-2 variants. Additionally, the increased sensitivity to M^{pro}-mediated cleavage of the N-ter-NB2E3 2× M^{pro} biosensor will be particularly useful in developing point-of-care testing (POCT) assays for detecting active SARS-COV-2 infection.

### Conclusion

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. A BRET-based M^{pro} biosensor comprising:
an mNeonGreen (mNG) reporter protein;
a NanoLuc (NLuc) reporter protein; and
x repeats of an N-terminal autocleavage peptide sequence of M^{pro};
wherein the x repeats of an N-terminal autocleavage peptide sequence of M^{pro} are located between the mNG reporter protein and the NLuc reporter protein.

2. The BRET-based M^{pro} biosensor of claim 1, wherein the x repeats of an N-terminal autocleavage peptide sequence of M^{pro} are two.

3. The BRET-based M^{pro} biosensor of claim 1, wherein the x repeats of an N-terminal autocleavage peptide sequence of M^{pro} are four.

4. The BRET-based M^{pro} biosensor of claim 1, wherein the x repeats of an N-terminal autocleavage peptide sequence of M^{pro} are eight.

5. The BRET-based M^{pro} biosensor of claim 1, wherein the N-terminal autocleavage peptide sequence of M^{pro} comprises a peptide sequence AVLQSGFR.

6. The BRET-based M^{pro} biosensor of claim 1, wherein the x repeats of an N-terminal autocleavage peptide sequence of M^{pro} are twelve.

7. The BRET-based M^{pro} biosensor of claim 6, wherein amino acid residues C-terminal to a critical Gln residue are varied.

8. The BRET-based M^{pro} biosensor of claim 7, wherein the twelve repeats of an N-terminal autocleavage peptide sequence of M^{pro} comprises Table 1 Sequence ID Listing No. 5.

9. A BRET-based M^{pro} biosensor comprising:
an mNeonGreen (mNG) reporter protein;
a NanoLuc (NLuc) reporter protein;
x repeats of an N-terminal autocleavage peptide sequence of M^{pro};
a first nanobody; and
a second nanobody;
wherein the x repeats of an N-terminal autocleavage peptide sequence of M^{pro} are located between the mNG reporter protein and the NLuc reporter protein.

10. The BRET-based M^{pro} biosensor of claim 9, wherein the first nanobody is NB1D10.

11. The BRET-based M^{pro} biosensor of claim 10, wherein the first nanobody is a C-terminal fusion.

12. The BRET-based M^{pro} biosensor of claim 10, wherein the first nanobody is an N-terminal fusion.

13. The BRET-based M^{pro} biosensor of claim 9, wherein the second nanobody is NB2E3.

14. The BRET-based M^{pro} biosensor of claim 13, wherein the second nanobody is a C-terminal fusion.

15. The BRET-based M^{pro} biosensor of claim 13, wherein the second nanobody is an N-terminal fusion.

16. The BRET-based M^{pro} biosensor of claim 9, wherein the x repeats of an N-terminal autocleavage peptide sequence of M^{pro} are two.

17. The BRET-based M^{pro} biosensor of claim 9, wherein the N-terminal autocleavage peptide sequence of M^{pro} comprises a peptide sequence AVLQSGFR.

18. A method of creating a BRET-based M^{pro} biosensor comprising:
placing x repeats of an N-terminal autocleavage peptide sequence of M^{pro} between an mNeonGreen (mNG) reporter protein and a NanoLuc (NLuc) reporter protein.

19. The method claim 18 further comprising, inserting a first nanobody between the nNG reporter protein and the x repeats of an N-terminal autocleavage peptide sequence of M^{pro}, and inserting a second nanobody between the x repeats of an N-terminal autocleavage peptide sequence of M^{pro} and the Nluc reporter protein.

20. The method of claim 18, wherein the x repeats of an N-terminal autocleavage peptide sequence of M^{pro} are two.
